(19) United States
(12) Patent Application Publication
(10) Pub. No.: US 2005/0209330 A1
(43) Pub. Date: Sep. 22, 2005

(54) Method of treatment of skin

(75) Inventor: Michael Kreindel, Haifa (IL)

Correspondence Address: BROWDY AND NEIMARK, P.L.L.C.;624 NINTH STREET, NW, SUITE 300, WASHINGTON, DC, 20001-5303, US

(73) Assignee: SYNERON MEDICAL LTD., Haifa (IL)

(21) Appl. No.: 10/805,496

(22) Filed: Mar. 22, 2004

(51) Int. Cl. A61K 7/135; A61K 31/195

(52) U.S. Cl. 514/561; 604/20

(57) ABSTRACT

A method for treating a region of skin. The method comprises applying 5-aminolevulinic acid (ALA) to the region of the skin and exposing the region to sunlight.

### FIELD OF THE INVENTION

This invention relates to methods for treating of skin.

### BACKGROUND OF THE INVENTION

5-Aminolevulinic acid (ALA) is a naturally occurring amino acid used in photodynamic therapy (PDT) of cancer and pre-cancer conditions such as actinic keratosis. ALA is applied to the skin surface to be treated in one or two applications of a 20% aqueous solution. After application of the ALA, the patient is advised to avoid exposure of the treated skin to sun light. 14 to 18 hours after application of the ALA, the patient returns to the care giver and the skin surface to be treated is exposed to blue light from a lamp for about 17 minutes.

In these treatments, a high concentration of ALA and high intensities of after application of ALA to the skin, the subject often experiences pain, irritation, and a strong erythemic reaction.

U.S. Pat. No. 5,474,528 to Meserol describes PDT treatment in which a patch with a photo-sensitizer is applied to a dermal lesion and controlled irradiation is carried out using optical energy produced by a light source.

U.S. Pat. No. 5,441,531 to Azrate et al., discloses PDT treatment in which light in the range of 600-700 nm produced by a lamp is used.

U.S. Pat. No. 5,489,279 to Meserol describes a sealed applicator for applying a photo-sensitizer such as ALA to skin for PDT treatment.

U.S. Pat. No. 5,856,566 to Golub discloses use of colored ALA where the color is imparted by irradiation of ALA crystals.

U.S. Pat. No. 5,707,401 to Talmore describes a device for simultaneous PDT treatment and hyperthermia.

U.S. Pat. No. 5,776,175 to Eckhouse et al., describes use of pulsed incoherent light for PDT treatment of tumors.

### SUMMARY OF THE INVENTION

The present invention provides a method for treating skin. In accordance with the method, ALA is applied to a skin region to be treated, and the skin region exposed to sunlight. The method of the invention may be used for cosmetic or therapeutic treatment of skin. Use of sunlight to irradiate the skin, as opposed to a specialized lamp, eliminates the need of the patient to return to the care giver after application of the ALA for irradiation treatment. Moreover, by using sunlight, the treated skin may be exposed to the light over a period of several days or weeks, with each daily exposure being half an hour or more. Thus, by irradiating with sunlight, a significantly longer exposure time can be obtained than when the irradiation must be performed by a care giver. In some applications, such as cosmetic treatment of the skin, a longer exposure time may allow a lower density of ALA to be applied to the skin. A lower concentration of ALA might reduce the pain and irritation experienced by the patient and as well as the erythemic reaction.

In a preferred embodiment the, the ALA is applied to the skin in the form of a cream. The cream preferably has a concentration of ALA lower than 20%. The cream is preferably applied to the skin in the morning to exposed skin regions (e.g., face, hand and neck, etc). The treated skin regions should preferably be exposed to sunlight for at least half an hour a day, and more preferably, at least 2 hours a day. The skin is preferably exposed to the sunlight for at least 5 days, and more preferably for at least 10 days.

The ALA may be applied to the skin together with other active substances such as vitamins A, C and E, glycolic acid, or a moisturizer.

The method of the invention may be used in a cosmetic treatment of the skin. Interaction of the applied ALA with sunlight stimulates skin metabolism and rejuvenation and kills bacteria on the skin surface preventing the appearance of acne. The cosmetic treatment may include, for example, skin bleaching, and pigmented lesion treatment.

The invention thus provides a method for treating a region of skin comprising:
(a) applying 5-aminolevulinic acid (ALA) to the region of the skin; and
(b) exposing the region to sunlight.

1. A method for treating a region of skin comprising:
(a) applying 5-aminolevulinic acid (ALA) to the region of the skin; and
(b) exposing the region to sunlight.

2. The method according to claim 1 wherein the region is exposed to sunlight for at least half an hour on each of a number of days.

3. The method according to claim 2 wherein the region is exposed to sunlight for at least two hours on each of a number of days.

4. The method according to claim 2 wherein the number of days is at least 5.

5. The method according to claim 2 wherein the number of days is at least 10.

6. The method according to claim 1 wherein the ALA is applied to the skin in the form of a cream.

7. The method according to claim 1 wherein the ALA is applied to the skin together with at least one other active ingredient.

8. The method according to claim 7 wherein the active ingredient is selected from the group comprising vitamin C, Vitamin A, Vitamin E, glycolic acid, and moisturizer.

9. The method according to claim 1 for use in cosmetic treatment of skin.

10. The method according to claim 9 wherein the cosmetic treatment includes one or more of the cosmetic treatments selected from the group comprising skin rejuvenation, acne treatment, skin bleaching, and pigmented lesion treatment.
